(19) United States
(12) Patent Application Publication
(10) Pub. No.: US 2024/0293112 A1
(43) Pub. Date: Sep. 5, 2024

(54) HYDROPHOBIC COATING FOR MEDICAL DEVICES

(71) Applicant: GYRUS ACMI, INC. D/B/A OLYMPUS SURGICAL TECHNOLOGIES AMERICA, Westborough, MA (US)

(72) Inventors: Tsunetaka Akagane, Hachioji-shi, Tokyo (JP); Issei Maeda, Tokyo (JP); Kester Julian Batchelor, Mound, MN (US); Teo Heng Jimmy Yang, Heath (GB); Takeshi Onaga, Medina, MN (US)

(21) Appl. No.: 18/578,584

(22) PCT Filed: Jul. 11, 2022
(86) PCT No.: PCT/US2022/073594

§ 371 Date: Jan. 11, 2024

Related U.S. Application Data
(60) Provisional application No. 63/203,176, filed on Jul. 12, 2021.

(51) Int. Cl. A61B 17/00; A61L 31/06; A61L 31/10; A61L 31/14

(52) U.S. Cl. CPC A61B 17/00; A61L 31/06; A61L 31/10; A61L 31/14; A61B 2017/00938

(57) ABSTRACT

A medical device and associated methods are disclosed. In one example, the medical device includes a hydrophobic coating. Hydrophobic coatings are shown that include a substrate bonding molecule chain, a hydrophobic molecule bonded to a first end of the substrate bonding molecule chain, and a reactive end bonded to a second end of the substrate bonding molecule chain. In selected examples the substrate bonding molecule chains are bonded to each other in a bond region adjacent to a surface of the component.

### CLAIM OF PRIORITY

This patent application claims the benefit of priority, under 35 U.S.C. § 119(e), to U.S. Provisional Patent Application Ser. No. 63/203,176, entitled “HYDROPHOBIC COATING AND METHOD FOR ELECTROSURGICAL DEVICES,” filed on Jul. 12, 2021, which is hereby incorporated by reference herein in its entirety.

### TECHNICAL FIELD

Embodiments described herein generally relate to medical devices. Specific examples of medical devices include electrosurgical devices.

### BACKGROUND

Several medical devices will benefit from a reduction in adhesion of material to one or more surfaces. For example, in medical cutting devices coagulation and/or tissue may adhere to a cutting assembly, and reduce the efficacy of a cutting operation. In particular, electrosurgical devices may have precise requirements for delivery of energy to tissue, and providing reduction in adhesion without sacrificing electrical properties is a technical challenge. Improved medical cutting devices and other medical devices with reduced adhesion surfaces are desired.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A shows stages of hydrophilic to superhydrophobic contact angles according to one example.

FIG. 1B shows a Cassie's state of hydrophobic pillars according to one example.

FIG. 1C shows a Wenzel's state of hydrophobic pillars according to one example.

FIG. 2 shows a coating molecule according to one example.

FIG. 3 shows a portion of a hydrophobic coating according to one example.

FIG. 4 shows electrical surface impedance of uncoated steel compared to HMDSO at about 85 nm compared to a hydrophobic coating according to one example at 1000 nm for a single laparoscopic RF vessel sealing device jaw.

FIG. 5 shows a monopolar colpotomy device according to one example.

FIG. 6 shows a bipolar vessel sealing device according to one example.

FIG. 7 shows a monopolar pencil according to one example.

FIG. 8 shows an ultrasonic device according to one example.

FIG. 9 shows a combined bipolar RF and ultrasonic device according to one example.

FIG. 10 shows lesion creating devices according to one example.

FIG. 11 shows a catheter device according to one example.

FIG. 12 shows a solid organ resection device according to one example.

### DETAILED DESCRIPTION

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

For simplicity, a bipolar RF energy delivery device example is used as an indicator of one of the more technically challenging variants that can incorporate this invention, but is not intended to be limited to this. This technology could be equally implemented on RF monopolar cutting devices, ultrasonic sealing devices, microwave ablation devices and variants of these that achieve the aforementioned tissue modification using these energy delivery modalities.

Example devices that will benefit from hydrophobic coatings, apart from an RF energy device, include any medical device where tissue sticking, or fluid adhesion is an issue. Example devices include, but are not limited to, visualization devices such as endoscopes, duodenoscopes, bronchoscopes, etc. Example devices include, but are not limited to, mechanical devices such as lithotripsy devices, etc. Example devices include, but are not limited to, cutting devices, such as bladed devices, etc. Example devices include, but are not limited to, energy devices other than RF energy devices, such as ablation devices, laser devices, resistive heating devices, etc.

One example is a bipolar RF device, including at least one tissue sealing plate including a non-stick coating, configured to reduce the sticking of soft tissue to the sealing plate during application of RF energy.

According to an embodiment of the present disclosure, an electrosurgical instrument is provided and includes at least one jaw member having an electrically conductive tissue sealing plate, configured to operably couple to a source of electrosurgical energy for treating tissue and a hydrophobic coating having a thickness of from about 35 nm to 2000 nm disposed on at least a portion of the tissue sealing plate. In one example, coating molecules as described provide a particularly beneficial combination of properties in a coating—being highly mechanically robust, and also highly hydrophobic as a result of a combination of molecule components described below. Surgical devices with coatings as described may be used multiple times without the coating degrading significantly.

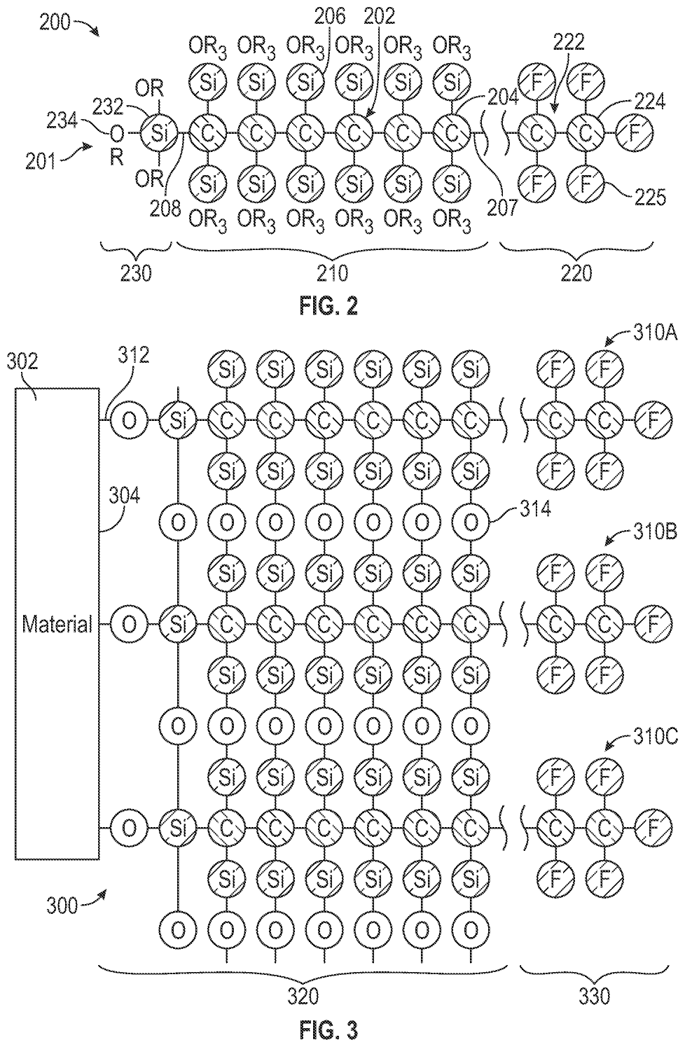

FIG. 2 shows one example of a coating molecule 200 that may be used to form a hydrophobic coating. In the example of FIG. 2, the coating molecule 200 includes a substrate bonding molecule chain 210. The coating molecule 200 also includes a hydrophobic molecule 220 bonded to a first end 207 of the substrate bonding molecule chain 210. The coating molecule 200 also includes a reactive end 230 bonded to a second end 208 of the substrate bonding molecule chain 210.

In one example, the substrate bonding molecule chain 210 includes a backbone 202. In the example of FIG. 2, the backbone 202 includes carbon atoms 204. In the example shown, the entire backbone 202 includes carbon atoms 204 although the invention is not so limited. Other backbones may be formed from silicon atoms, alternating silicon and oxygen atoms, or other suitable backbone chemistries.

In one example, the substrate bonding molecule chain 210 includes silicon side groups 206. In the example of FIG. 2, the silicon side groups may further include oxygen and R3 groups bonded to the silicon. As described in more detail below in reference to FIG. 3, the side groups 206 on the substrate bonding molecule chain 210 may be used to form intermolecular bonds between adjacent substrate bonding molecule chains 210. Although the example of FIG. 2 shows siloxane (silicon and oxygen) groups, other side groups capable of bonding to adjacent substrate bonding molecule chains 210 are also within the scope of the invention.

In one example, the hydrophobic molecule 220 includes a backbone 222. Similar to the description of the substrate bonding molecule chain 210, the backbone 222 of the hydrophobic molecule 220 may include carbon atoms 224 as shown, or other backbone chemistries such as silicon, siloxane, etc. In the example of FIG. 2, the hydrophobic molecule 220 includes side groups 225. In the example shown, the side groups are fluorine atoms. Other examples of side groups that provide good hydrophobic properties may include methyl groups or other hydrophobic side groups.

In one example the hydrophobic molecule 220 is a fluoropolymer. The hydrophobic molecule 220 shown in FIG. 2 illustrates a short backbone 222 of two carbons. In one example despite the short backbone, the two carbon hydrophobic molecule 220 is referred to as a fluoropolymer. Other lengths of backbone 222 are also within the scope of the invention.

In one example, the substrate bonding molecule chain 210 is longer than the hydrophobic molecule 220. The relative length of the substrate bonding molecule chain 210 compared to the hydrophobic molecule 220 provides a number of advantages. In one example, a relatively long substrate bonding molecule chain 210 facilitates bonding between adjacent substrate bonding molecule chains 210 in other coating molecules as illustrated in FIG. 3. A longer substrate bonding molecule chain 210 provides a more mechanically robust coating due to increased strength between coating molecules 200.

A relatively shorter hydrophobic molecule 220 is also more mechanically robust. Longer hydrophobic molecules 220 may be more prone to breaking free from the substrate bonding molecule chain 210, or breaking along their backbone 222. The combination of a substrate bonding molecule chain 210 that is longer than the hydrophobic molecule 220 provides both the properties of increased strength between coating molecules 200 and shorter, more robust hydrophobic molecules 220.

In one example the reactive end 230 includes a silicon atom 232. In the example shown, an oxygen atom 234 is located at an exposed end 201 of the reactive end 230 to bond with a surface of a component of a surgical instrument, although the invention is not so limited. Other reactive end 230 chemistries are within the scope of the invention, and may depend on the surgical device component material that a hydrophobic coating is being bonded to.

FIG. 3 shows a portion of a coating 300. A substrate 302 is shown that may be part of any number of surgical devices. Selected examples of components with substrates 302 include but are not limited to, RF electrodes in electrosurgery devices, monopolar electrodes in electrosurgery devices, cutting blades, exteriors or interiors of catheters, etc. As noted above, other examples of substrates 302, include any medical device where tissue sticking, or fluid adhesion is an issue. Example devices include, but are not limited to, visualization devices such as endoscopes, duodenoscopes, bronchoscopes, etc. Example devices include, but are not limited to, mechanical devices such as lithotripsy devices, etc. Example devices include, but are not limited to, cutting devices, such as bladed devices, etc. Example devices include, but are not limited to, energy devices other than RF energy devices, such as ablation devices, laser devices, resistive heating devices, etc.

FIG. 3 shows three coating molecules 310A, 310B, 310C similar to the coating molecule 200 from FIG. 2. The three coating molecules 310A, 310B, 310C each are shown including a substrate bonding molecule chain and a hydrophobic molecule bonded to a first end of the substrate bonding molecule chain. A reactive end is shown bonding molecule 310A to a surface 304 of the component substrate 302 at bonding sites 312. The coating molecules 310A, 310B, 310C are further shown bonded to each other at bonding sites 314. In the example shown, the bonding sites 314 include siloxane bonds, although the invention is not so limited.

In one example, the surface 304 may be modified prior to application of the coating, although the invention is not so limited. Examples of surface modification may include sand blasting or etching to roughen the surface and enhance adhesion. In one example, the inventors discovered that #1000 grit sandblasting, which is a grit size not normally used in preparing such surfaces and coating materials, can be used to roughen a surface before coating. In one example, the substrate 302 includes ceramic material, and other large grit sizes may damage the ceramic substrate 302. Sandblasting using small grit size as described also provides a level of final product dimensional control that larger grit sizes do not provide. In one example, sandblasting grit used for surface roughening is between #800 and #1200. In one example, sandblasting grit used for surface roughening is between #900 and #1100. In one example, sandblasting grit used for surface roughening is between #950 and #1050. Surface roughness modification may increase surface area to strengthen a bond. Surface roughness modification may also provide mechanical structure that enhances adhesion of a coating.

Any amount of bonding between coating molecules 310A, 310B, 310C will enhance a mechanical strength of a resulting coating. As discussed above, a relatively long substrate bonding molecule chain facilitates larger amounts of bonding between adjacent substrate bonding molecule chains in adjacent coating molecules 310A, 310B, 310C. By using only the substrate bonding molecule chains 210 for inter molecule bonding between coating molecules, the hydrophobic molecules may be exclusively used for hydrophobic properties. For example, all side groups 225 as illustrated in FIG. 2 may include fluorine atoms to provide high hydrophobicity.

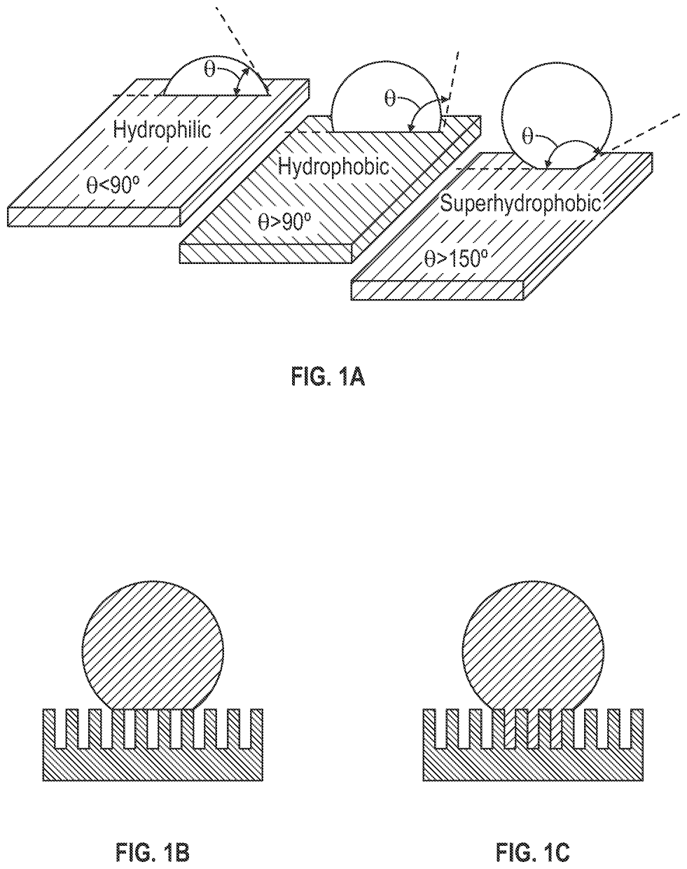

In selected examples, in addition to chemical hydrophobicity related to surface energy, coatings may also include physical structure that imparts additional hydrophobicity. Examples of ultrahydrophobic physical structure is illustrated in FIGS. 1A-1C, and discussed in more detail below. In selected examples, ultrahydrophobic physical structure may result from molecular structure. In other selected examples, ultrahydrophobic physical structure may result from surface modification. Examples of surface modification may include etching, laser patterning, or other mechanisms to form physical surface structure.

In one example, the hydrophobic coating is applied by dip coating, although the invention is not so limited. Other examples of coating methods include spraying a single coat, spraying multiple coats, dip coating multiple coats, other painting applications, chemical vapor deposition, physical vapor deposition, etc. One of ordinary skill in the art will recognize that different methods of deposition will result in different physical characteristics in a final coating such as coating uniformity, thickness variation, etc. that are detectable upon inspection. In one aspect of the present disclosure, the hydrophobic coating has a thickness of about 1000 nm. In one example, the hydrophobic coating has a thickness in a range from 1 nm to 1000 nm. In one example, the hydrophobic coating has a thickness in a range from 2 nm to 600 nm. In another aspect of the present disclosure, the hydrophobic coating has a substantially uniform thickness. In another aspect of the present disclosure, the hydrophobic coating has a non-uniform thickness. Thicknesses and thickness ranges detailed above provide hydrophobic non-stick properties and mechanical strength to the coatings, while also providing acceptable electrical properties that thicker coating may not provide. The inventors have discovered that this combination of properties can be advantageous for surgical devices described in the present disclosure.

In another aspect of the present disclosure, the hydrophobic coating is discontinuous. In another aspect of the present disclosure, the hydrophobic coating is continuous. In another aspect of the present disclosure, the electrosurgical instrument includes an insulative layer disposed on at least a portion of the tissue sealing plate. In another aspect of the present disclosure, the hydrophobic coating is disposed on at least a portion of each of the opposing jaw members. In another aspect of the present disclosure, the tissue sealing plate is formed of stainless steel. In another aspect of the present disclosure, the hydrophobic coating is disposed on the support base of the jaw and not on the tissue sealing plate. In another aspect of the present disclosure, the hydrophobic coating is disposed on at least a portion of the support base of the jaw and the tissue sealing plates. In another aspect of the present disclosure, the hydrophobic coating is disposed at least partially on the support base of the jaw, the tissue sealing plate and the shorting preventing ‘stops’ associated with the tissue sealing plates. In another aspect of the present disclosure, the hydrophobic coating is disposed at least partially on one or all of the shorting preventing ‘stops’ associated with the tissue sealing plates.

According to another embodiment of the present disclosure, an electrosurgical instrument is provided and includes a pair of opposing jaw members. Each of the opposing jaw members includes an electrically conductive tissue sealing plate configured to operably couple to a source of electrosurgical energy, for treating tissue, a support base configured to support the tissue sealing plate, and an insulative housing configured to secure the tissue sealing plate to the support base.

A hydrophobic coating having a thickness of from about 35 nm to 2000 nm is disposed on at least a portion of at least one of the opposing jaw members.

In one aspect of the present disclosure, the hydrophobic coating is disposed on at least a portion of each of the sealing plates, the support base and the insulative housing. In another aspect of the present disclosure, the hydrophobic coating has a thickness of about 1000 nm. In another aspect of the present disclosure, the hydrophobic coating has a substantially uniform thickness. In another aspect of the present disclosure, the hydrophobic coating has a non-uniform thickness.

In another aspect of the present disclosure, the hydrophobic coating is discontinuous. In another aspect of the present disclosure, the hydrophobic coating is continuous.

According to another embodiment of the present disclosure, an electrically conductive tissue sealing plate is provided and includes a stainless steel layer having a first surface and an opposing second surface. The stainless steel layer is configured to deliver energy to tissue. An insulative layer is disposed on the surface of the stainless steel layer and a hydrophobic coating having a thickness of from about 35 nm to about 2000 nm is disposed on at least a portion of the first surface of the stainless steel layer.

In one aspect of the present disclosure, the hydrophobic coating has a thickness of about 1000 nm. In another aspect of the present disclosure, the hydrophobic coating has a non-uniform thickness. In another aspect of the present disclosure, the hydrophobic coating is discontinuous.

According to another embodiment of the present disclosure, a method of inhibiting tissue from sticking to an electrically conductive component of an electrosurgical tissue sealing device during application of energy to tissue is provided. The method includes applying a hydrophobic coating on at least one portion of an electrically conductive component of an electrosurgical tissue sealing device using a dipping technique. The substrate or the surface receiving the coating may be treated such as sandblasted or chemically treatment to improve coating adhesion. The method includes applying a hydrophobic coating on at least one portion of an electrically conductive component of an electrosurgical tissue sealing device using a spraying technique. The method includes applying a hydrophobic coating on at least one portion of an electrically conductive component of an electrosurgical tissue sealing device using a painting technique. The method can include controlling a thickness of the polytetrafluoroethylene coating applied from about 35 nm to 2000 nm. Another possible technique of coating may be spin coating to achieve a uniform thickness. The coating process may undergo a raised temperature curing e.g., oven, to obtain a solidified state.

According to another embodiment of the present disclosure, an electrosurgical instrument is provided and includes a pair of jaw members, each having an electrically conductive sealing plate configured to operably couple to a source of electrosurgical energy. The tissue sealing plates are configured to deliver electrosurgical energy to tissue based on at least one sensed tissue parameter. The electrosurgical instrument includes a non-stick coating disposed on at least a portion of each tissue sealing plate. The non-stick coating has a thickness controlled to reduce sticking of the electrically conductive sealing plates during the delivery of electrosurgical energy to the tissue and to permit a sensing of the at least one tissue parameter. In one aspect of the present disclosure the hydrophobic coating has a thickness of from about 35 nm to about 2000 nm is disposed on at least a portion of the first surface of the stainless steel layer. In one aspect of the present disclosure, the hydrophobic coating has a thickness of about 1000 nm. In another aspect of the present disclosure, the hydrophobic coating has a non-uniform thickness. In another aspect of the present disclosure, the hydrophobic coating is discontinuous. In another aspect of the present disclosure, the at least one tissue parameter is selected from the group consisting of temperature, resistance, light and impedance.

According to another embodiment of the present disclosure, a method of inhibiting tissue from sticking to an electrically conductive component of an electrosurgical tissue sealing device during application of energy to tissue is provided.

Particular aspects of the present electrosurgical tissue sealing instruments are described herein below with reference to the accompanying drawings; however, it is to be understood that the disclosed aspects are merely examples of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the concepts of the present disclosure in virtually any appropriately detailed structure.

Like reference numerals may refer to similar or identical elements throughout the description of the figures. As shown in the drawings and described throughout the following description, as is traditional when referring to relative positioning on a surgical instrument, the term “proximal” refers to the end of the apparatus which is closer to the user and the term “distal” refers to the end of the apparatus which is further away from the user. The term “clinician” refers to any medical professional (i.e., doctor, surgeon, nurse, or the like) performing a medical procedure involving the use of aspects described herein.

As described in more detail below with reference to the accompanying figures, the present disclosure is directed to electrosurgical instruments having a non-stick coating disposed on one or more components (e.g., tissue sealing plates, jaw members, electrical leads, insulators etc.) The thickness of the non-stick coating is carefully controlled, allowing for desired electrical performance while providing tissue sticking reduction during tissue sealing.

Any material capable of providing the desired functionality (namely, reduction of tissue sticking while simultaneously maintaining sufficient electrical transmission to permit tissue sealing) may be used as the non-stick coating, provided it has adequate biocompatibility.

Previously disclosed materials include HMDSO that disclose the use of and application of HMDSO on similar energy delivery devices. In their disclosure they also include other materials such as TMDSO and the plasma deposition system to achieve non-stick performance on tissue contacting modification instruments.

These coatings when employed in such processes and in the thicknesses claimed, result in ‘Superhydrophobic’ coatings. Superhydrophobic coatings are different from typical low surface energy materials alone, as the function of these coatings requires the coating structure to be deposited in such a way to create Hydrophobic pillars that repel water, oils and blood, in a way not possible by low surface energy alone.

In presently known art, a hydrophilic coating is one with a water Contact Angle of less than 90°. A hydrophobic surface, is a surface with a water Contact Angle of between 90° and 150° and a superhydrophobic surface (also known as an ultrahydrophobic surface) is a surface that has a water contact angle of 150° and above. See FIGS. 1A-1C. HMDSO when applied to a surface can tend towards a Superhydrophobic state (depending on application settings) or at least above those typically achieved by the same materials being applied in a non-hydrophobic pillar creating process or other chemical assembly of these materials that do not combine to create this polymeric structure. FIG. 1B is an illustration of the ‘Cassie's State’ of hydrophobic pillars and FIG. 1C is an illustration of the ‘Wenzel's state’ of hydrophobic pillars.

Electrical properties may drive the usable thickness of a coating over a surface that is intended to be used to deliver electrical energy or receive electrical feedback from the contact tissue. While the thickness of the coating is important, it varies upon the electrical modality being employed. Higher frequencies of electrical AC output require different considerations to lower frequencies. In the examples provided, the thickness of coating used for microwave transmission is different to that required for Radio Frequency (RF) transmission and this is different again to other energy modalities, such as ultrasonic modalities, when vibration transmission and absorption requires different coating thicknesses.

The example used in this disclosure is a pair of jaw members, each having an electrically conductive sealing plate configured to operably couple to a source of electrosurgical energy. The tissue sealing plates are configured to deliver electrosurgical energy to tissue based on at least one sensed tissue parameter. The electrosurgical instrument includes a non-stick coating disposed on at least a portion of each tissue sealing plate. The non-stick coating has a thickness controlled to reduce sticking of the electrically conductive sealing plates during the delivery of electrosurgical energy to the tissue and to permit a sensing of the at least one tissue parameter. In one aspect of the present disclosure the hydrophobic coating has a thickness of from about 35 nm to about 2000 nm is disposed on at least a portion of the first surface of the stainless steel layer. In one aspect of the present disclosure, the hydrophobic coating has a thickness of about 1000 nm. In another aspect of the present disclosure, the hydrophobic coating has a non-uniform thickness. In another aspect of the present disclosure, the hydrophobic coating is discontinuous. In another aspect of the present disclosure, the at least one tissue parameter is selected from the group consisting of temperature, resistance, light and impedance.

The limitations of the thickness parameters are in this case limited by the desired ability to deliver RF electrical energy as well as, in some cases, gain feedback through such coating. Thicknesses of about 2000 nm and above become too significant and present losses of fidelity in energy delivery and feedback that are undesirable and unnecessary considering the predicted normal usage life of such a device.

At around 1000 nm, the disclosed hydrophobic coating has an acceptable level of electrical performance while providing hydrophobic, or non-stick properties that enhance clinical procedural use.

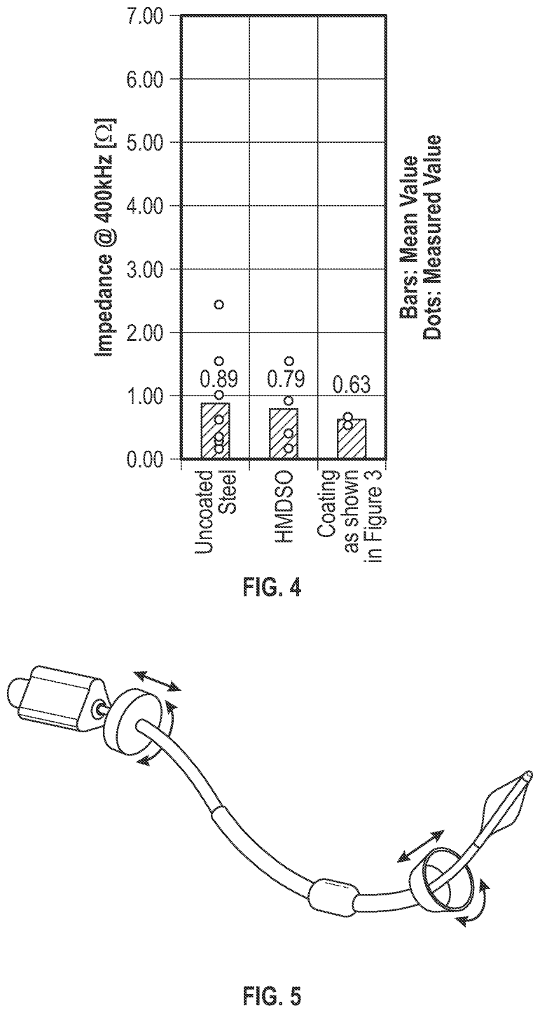

FIG. 4 compares the electrical performance of 1) an uncoated steel electrically conductive sealing plate (with surface impurities and contaminates typical of production methods of a device of this type), 2) an about 85 nm thick HMDSO coating of a steel electrically conductive sealing plate and 3) an about 1000 nm thick hydrophobic coating as shown in FIG. 3, at 400 khz (RF) frequency.

Those knowledgeable in the art, will now understand how these thicknesses will need to be modified according to the wavelength (frequency) of the energy being supplied and how the hydrophobic coating relates electrically to previously disclosed non-stick HMDSO technology and no coating.

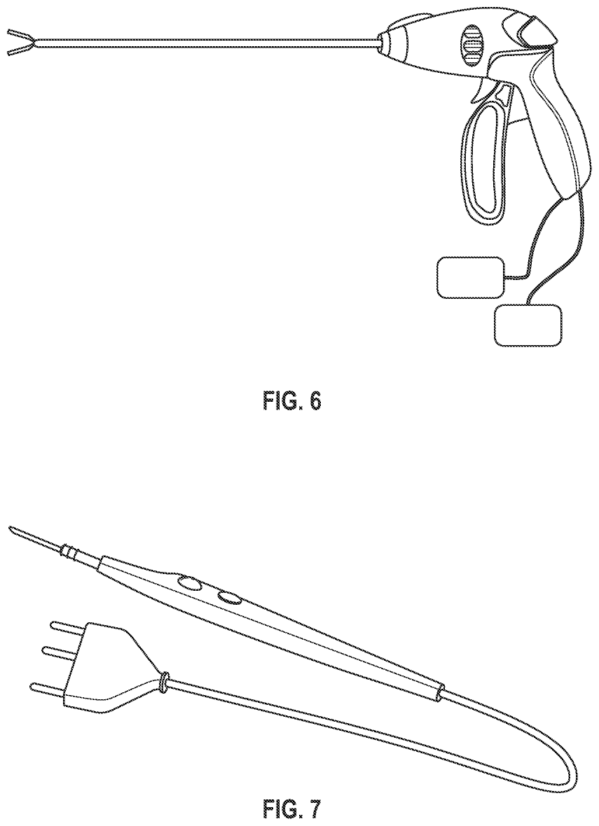

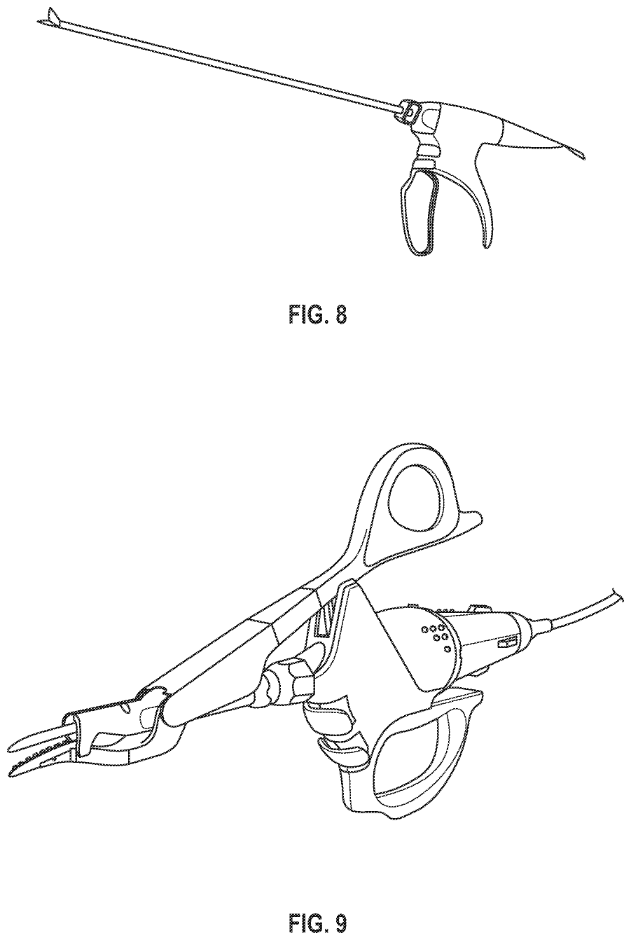

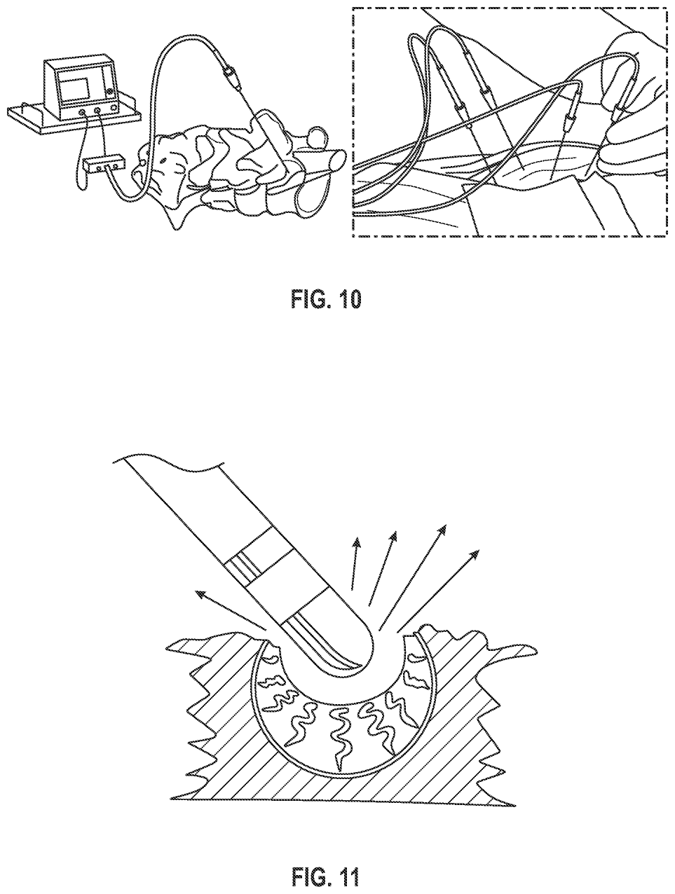

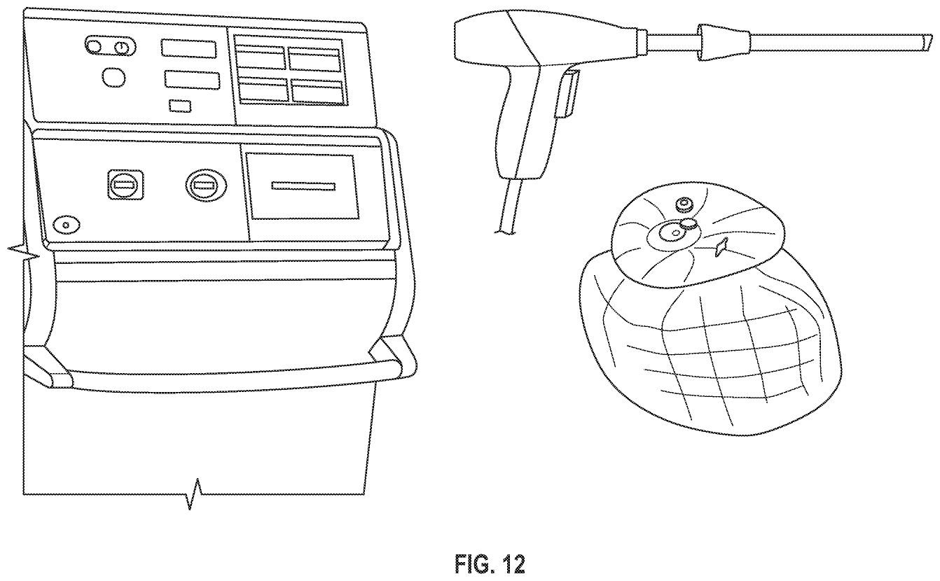

FIGS. 5 through 12 include other devices that may incorporate the hydrophobic coating and are included to demonstrate the variety of energy delivery and tissue modification device type that can be improved upon through the inclusion of this type of technology. In these examples it is expected that the hydrophobic coating is disposed in part or completely on the energy application element and optionally in the surrounding device structure where tissue contact may occur. Either partially or completely and in thicknesses as described previously in this disclosure.

To better illustrate the method and apparatuses disclosed herein, a non-limiting list of embodiments is provided here:

Example 1 includes a surgical device. The surgical device includes a surgical device component, and a coating at least partially covering the component of the surgical device, wherein the coating is configured according to an example of the present disclosure.

Example 2 includes a surgical device. The surgical device includes a surgical device component and a hydrophobic coating at least partially covering the component of the surgical device. The hydrophobic coating is formed from coating molecules including a substrate bonding molecule chain, a hydrophobic molecule bonded to a first end of the substrate bonding molecule chain and a reactive end bonded to a second end of the substrate bonding molecule chain, wherein the substrate bonding molecule chain is longer than the hydrophobic molecule.

Example 3 includes the surgical device of example 2, wherein the substrate bonding molecule chain includes a carbon atom backbone.

Example 4 includes the surgical device of any one of examples 2-3, wherein the substrate bonding molecule chain includes a silicon atom backbone.

Example 5 includes the surgical device of any one of examples 2-4, wherein the hydrophobic molecule includes a fluoropolymer.

Example 6 includes the surgical device of any one of examples 2-5, wherein the hydrophobic molecule includes a siloxane backbone.

Example 7 includes a surgical device. The surgical device includes a surgical device component and a hydrophobic coating at least partially covering the component of the surgical device. The hydrophobic coating is formed from coating molecules including a substrate bonding molecule chain, a hydrophobic molecule bonded to a first end of the substrate bonding molecule chain and a reactive end bonded to a second end of the substrate bonding molecule chain, wherein substrate bonding molecule chains are bonded to a surface of the component at the reactive end, and bonded to each other in a bond region adjacent to the surface of the component.

Example 8 includes the surgical device of example 7, wherein the substrate bonding molecule chain includes a silicon atom backbone.

Example 9 includes the surgical device of any one of examples 7-8, wherein the substrate bonding molecule chain includes a carbon atom backbone.

Example 10 includes the surgical device of any one of examples 7-9, wherein the substrate bonding molecule chains are bonded to each other with siloxane bonds.

Example 11 includes the surgical device of any one of examples 7-10, wherein the substrate bonding molecule chains are bonded to the substrate with siloxane bonds.

Example 12 includes the surgical device of any one of examples 7-11, wherein the hydrophobic molecule includes a fluoropolymer.

Example 13 includes the surgical device of any one of examples 7-12, wherein the substrate bonding molecule chain is longer than the hydrophobic molecule.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. A surgical device, comprising:
a surgical device component;
a hydrophobic coating at least partially covering the component of the surgical device, wherein the hydrophobic coating is formed from coating molecules including;
a substrate bonding molecule chain;
a hydrophobic molecule bonded to a first end of the substrate bonding molecule chain;
a reactive end bonded to a second end of the substrate bonding molecule chain;
wherein the substrate bonding molecule chain is longer than the hydrophobic molecule.

2. The surgical device of claim 1, wherein the substrate bonding molecule chain includes a carbon atom backbone.

3. The surgical device of claim 1, wherein the substrate bonding molecule chain includes a silicon atom backbone.

4. The surgical device of claim 1, wherein the hydrophobic molecule includes a fluoropolymer.

5. The surgical device of claim 1, wherein the hydrophobic molecule includes a siloxane backbone.

6. A surgical device, comprising:
a surgical device component;
a hydrophobic coating at least partially covering the component of the surgical device, wherein the hydrophobic coating is formed from coating molecules including;
a substrate bonding molecule chain;
a hydrophobic molecule bonded to a first end of the substrate bonding molecule chain;
a reactive end bonded to a second end of the substrate bonding molecule chain;
wherein substrate bonding molecule chains are bonded to a surface of the component at the reactive end, and bonded to each other in a bond region adjacent to the surface of the component.

7. The surgical device of claim 6, wherein the substrate bonding molecule chain includes a silicon atom backbone.

8. The surgical device of claim 6, wherein the substrate bonding molecule chain includes a carbon atom backbone.

9. The surgical device of claim 6, wherein the substrate bonding molecule chains are bonded to each other with siloxane bonds.

10. The surgical device of claim 9, wherein the substrate bonding molecule chains are bonded to the substrate with siloxane bonds.

11. The surgical device of claim 10, wherein the hydrophobic molecule includes a fluoropolymer.

12. The surgical device of claim 11, wherein the substrate bonding molecule chain is longer than the hydrophobic molecule.
